(19) United States
(12) Patent Application Publication
(10) Pub. No.: US 2007/0072910 A1
(43) Pub. Date: Mar. 29, 2007

(54) Compositions and methods for lowering plasma concentrations of low density lipoproteins in humans

(76) Inventor: Michael L. Smith, Phoenix, AZ (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/322,115

(22) Filed: Dec. 29, 2005

Related U.S. Application Data
(60) Provisional application No. 60/722,716, filed on Sep. 29, 2005.

(51) Int. Cl. A61K 31/455; A61K 31/22; A61K 31/205

(52) U.S. Cl. 514/332; 514/356; 514/337; 514/546; 514/554

(57) ABSTRACT

The present invention is directed to compositions and method to lower the plasma concentration of a low density lipoprotein in a human. One aspect is the invention is directed to compositions comprising at least one pharmaceutically acceptable L-carnitine compound and at least one pharmaceutically acceptable niacin compound, in such amount as to reduce the concentration of a low density lipoprotein. Another embodiment is directed to a composition of L-carnitine and niacin that substantially reduces side-effects associated with niacin treatment. Another aspect of the invention relates to a method for treating hyperlipidemia while substantially reducing side-effects associated with niacin treatment by administering a composition of L-carnitine and niacin.

### CROSS REFERENCE TO RELATED APPLICATIONS

This application claims priority under 35 USC § 119(e) from U.S. Provisional Application Ser. No. 60/722,716, filed Sep. 29, 2005, the entirety of which is incorporated herein by reference.

### FIELD OF THE INVENTION

This invention relates to compositions and methods for reducing levels of certain low density lipoproteins in humans thereby improving cardiovascular health. This invention further relates to reducing or eliminating potential side-effects from a traditional treatment of niacin to reduce the levels of low density lipoproteins in humans.

### BACKGROUND OF THE INVENTION

Heart disease remains the leading cause of death in the United States, despite the efforts of the medical community over the past several decades, with millions of heart attacks annually and nearly one million deaths per year. The risk for heart disease is commonly linked to the plasma level of cholesterol, with higher levels indicating a greater risk of heart attack. Not all cholesterol forms are thought to be directly linked to heart attacks.

Lipoproteins transport cholesterol and triglycerides to and from tissue cells. Generally, the higher the percentage of lipid in the lipoproteins, the lower the density; conversely, the greater the proportion of protein, the higher the density. High density lipoproteins (HDLs) are thought to play a beneficial role in human circulatory health. HDLs transport excess cholesterol from peripheral tissue cells to the liver for degradation into bile. Another type of cholesterol containing protein, termed low density lipoproteins (LDLs), are thought to be a health risk because they transport cholesterol to peripheral tissues, making it available for hormone synthesis. High levels of LDLs indicate that excess cholesterol deposits are being made in the arterial walls. Very low density lipoproteins mainly deposit cholesterol in adipose tissue, so they are not thought to be as dangerous as LDLs.

One type of LDL commonly found in human plasma is called lipoprotein(a) [hereinafter abbreviated as “Lp(a)”]. This is a lipoprotein containing cholesterol consisting of a low density lipid core which is encased in a protein shell. This outer protein is called apo(a). The exact nature of apo(a) is genetically determined through a dominant trait and seems fixed from birth through the remainder of life. There seems to be over 20 different forms of this protein commonly observed in any human population. These forms differ from one another in a step-wise manner, in that the coat protein, apo(a), differs in small but significant increments of molecular weight between individuals. A human carries but one form of this coat protein with a characteristic molecular weight.

Levels of human Lp(a) may be observed from 0 to 100 mg/dL, when tested with a robust technique able to report all forms of this lipoprotein. A typical level of Lp(a) for Caucasians and Asians is 10 mg/dL and for African-Americans is about 20 mg/dL. A strong correlation has been made between high plasma levels of Lp(a) and heart attacks. Levels of this lipoprotein above 30 mg/dL are thought to increase the possibility of a heart attack by up to 70% over the general population. This correlation is much stronger than that between overall cholesterol levels and heart attack. It is estimated that perhaps up to 30% of certain populations exhibit higher levels of Lp(a) than may be deemed safe.

Lp(a) is metabolized and regulated via the liver. Recognition by the liver of Lp(a) is highly dependent upon the molecular weight of the coat protein. Individuals with apo(a) coat protein of higher than usual molecular weights tend to have lower than normal levels of Lp(a) and people with low molecular weight coat protein tend to have higher than normal levels of Lp(a). For this reason, the level of Lp(a) is high in these individuals who carry the low molecular weight apo(a) and eventually results in blood vessel blockage and heart attack. Up to now, changes of diet or addition to diet has not been observed to significantly affect the level of Lp(a) in human plasma.

Since increased levels of LDLs are considered to play a role in atherosclerosis and hyperlipidemia, and thus myocardial infarctions, strokes and peripheral vascular disease, it is desirable to find a pharmaceutical composition and treatment that may reduce LDLs, specifically Lp(a). One treatment currently used to lower total plasma cholesterol levels, including Lp(a), is the use of oral niacin. In order to significantly lower total cholesterol and low density lipoproteins, high daily doses of niacin must be administered. This amount of niacin is considered very uncomfortable by many patients due to side effects, such that up to 40% of the patients on a niacin regimen will not comply with the regimen and may cease use of niacin therapy altogether.

Immediate release formulations of niacin release their entire product within about 30 to 60 minutes following ingestion. While this is somewhat effective in reducing total cholesterol, LDL cholesterol, triglycerides, and Lp(a), this treatment is usually associated with undesirable side-effects. High doses of niacin may result in liver damage, peptic ulcers, skin rashes, and increased glucose and uric acid levels in the blood. Normal doses result in a high incidence of skin flushing. Treating hyperlipidemia over the course of several doses of niacin often results in uncomfortable flushing after each dose. To alleviate these side effects, sustained release formulations of nicotinic acid were developed to slowly release the drug into the blood stream. However, these formulations are not as beneficial as immediate release formulations in reducing lipoprotein concentration. Sustained release formulations have also resulted in a greater incidence of liver toxicity. These side-effects of both immediate release and sustained release formulations are thought to contribute heavily to the high drop-out rate of patients on a niacin regimen.

### SUMMARY OF THE INVENTION

In accordance with one embodiment, there is provided a composition suitable for the treatment and prevention of cardiovascular disease comprising L-carnitine and niacin, or acceptable substitutes and derivatives thereof. A pharmaceutically acceptable L-carnitine compound may also be a salt and/or ester thereof. A pharmaceutically acceptable niacin compound may be any source of nicotinic acid or niacinamide, including the free bases, their salts, their anions, or mixtures thereof. The active ingredients are present in such amounts as to reduce plasma levels of total cholesterol, LDL cholesterol, and/or Lp(a). In one embodiment, plasma levels of HDLs are increased.

In accordance with a further embodiment, there is provided a method comprising administering at least once daily to a human a composition suitable for the treatment and prevention of cardiovascular disease comprising (or in one embodiment, consisting essentially of) L-carnitine and niacin, or acceptable salts, esters, and/or derivatives thereof. An L-carnitine compound can also be an acceptable salt and/or ester thereof. At least one active ingredient of niacin can be any source of nicotinic acid or niacinamide, including the free bases, their salts, their anions or mixtures thereof. These same compositions and regiments may also be used for treatment or prevention of ischemic heart disease, atherosclerosis, arteriosclerosis, cerebrovascular disease, myocardial infarction, stroke, angina, and/or the like.

Another embodiment relates to a composition suitable for effectively reducing the concentration of an LDL in a human. This composition comprises (and in some embodiments consists essentially of) a pharmaceutically acceptable L-carnitine compound and a pharmaceutically acceptable niacin compound wherein the compounds are included in such amounts as to reduce the concentrations of a low density lipoprotein, such as Lp(a), in a human. Another embodiment relates to a composition that is suitable for effectively reducing the concentration of Lp(a). Another aspect relates to a composition suitable for effectively reducing the concentration of LDL-cholesterol. The pharmaceutically acceptable L-carnitine compound may also be an acceptable salt and/or ester, or a mixture thereof. The pharmaceutically acceptable niacin compound may be any source of nicotinic acid or niacinamide, including the free bases, their salts, their anions, or a mixture thereof.

Another aspect relates to a composition comprising pharmaceutically acceptable L-carnitine and pharmaceutically acceptable niacin, wherein the active ingredients of both compounds are in such amounts to reduce plasma levels of at least one low density lipoprotein and/or to not substantially cause side-effects of niacin treatment in a human. One embodiment substantially reduces the side-effects of a traditional niacin treatment. In one embodiment at least one active ingredient of the at least one pharmaceutically acceptable L-carnitine compound totals x moles and at least one active ingredient of the pharmaceutically acceptable niacin compound totals y moles and the mole ratio of x/y is preferably between 0.5 and 100. Otherwise stated, the total moles of L-carnitine, whether present as L-carnitine or a salt, ester or derivative thereof, totals x moles and the total moles of niacin, whether present as nicotinic acid, niacinamide or other derivative or form, totals y moles and the mole ratio of x/y is preferably between 0.5 and 100. For the sake of clarity, one mole niacinamide equals one mole niacin, one mole ascorbyl niacinamide equals one mole niacin, and one mole of inositol hexanicotinate equals six moles niacin. In another embodiment, the mole ratio of x/y is between 0.6 and 76, including between 3.0 and 16. These embodiments also have the effect of reducing the plasma concentration of a low density lipoprotein such as Lp(a) or LDL-cholesterol. Some embodiments may further include vitamins and/or minerals.

Another aspect relates to a composition suitable for lowering plasma levels of low density lipoproteins comprising a pharmaceutically acceptable L-carnitine compound and a pharmaceutically acceptable niacin compound that have a mutual synergistic effect of reducing plasma concentration of a low density lipoprotein in humans. In a preferred embodiment, this method reduces the levels of Lp(a). Another aspect relates to a method for reducing the plasma concentration of LDL-cholesterol.

Another embodiment relates to a method for effectively reducing the concentration of an LDL in a human. This method comprises administering at least once daily to a human a composition comprising (or in one embodiment, consisting essentially of) a pharmaceutically acceptable L-carnitine compound and a pharmaceutically acceptable niacin compound wherein the compounds are in such amounts as to reduce the concentrations of a low density lipoprotein in a human. A preferred embodiment relates to a method for effectively reducing the concentration of Lp(a). Another embodiment relates to a method for effectively reducing the concentration of LDL-cholesterol. The pharmaceutically acceptable L-carnitine compound may also be an acceptable salt and/or ester, or a mixture thereof. The pharmaceutically acceptable niacin compound may be any source of nicotinic acid or niacinamide, including the free bases, their salts, their anions, or a mixture thereof. A preferred embodiment of the method is when between 200-5000 mg/day of the active ingredient of a pharmaceutically acceptable L-carnitine compound is administered to the human. A preferred embodiment of the method is when between 50-240 mg/day of the active ingredient of a pharmaceutically acceptable niacin compound is administered to the human. Another embodiment of the method is when the composition is administered in multiple doses and the active ingredient of the pharmaceutically acceptable niacin compound totals 25-120 mg/dose.

Another embodiment relates to a method for reducing flushing and/or discomfort in an individual being treated for hyperlipidemia with niacin comprising administering at least once daily to a human a composition comprising a pharmaceutically acceptable L-carnitine compound with a pharmaceutically acceptable niacin compound in such amounts as to not substantially cause flushing or discomfort in the human. As compared to a traditional niacin regimen, there are substantially reduced flushing and discomfort. Another aspect of this method does not substantially cause hepatotoxicity in the human. Another aspect of this method does not substantially cause glucose and/or uric acid level elevation in blood of the human. Thus, in some embodiments, there are substantially reduced side effects of a traditional niacin treatment.

Another aspect of the present invention relates to a method for reducing the plasma concentration of a low density lipoprotein utilizing a composition consisting essentially of a pharmaceutically acceptable L-carnitine compound and a pharmaceutically acceptable niacin compound in such amount as to reduce the plasma concentration of a low density lipoprotein. Another aspect of this method does not substantially cause significant side-effects generally associated with niacin treatment. Thus, there are substantially reduced side effects as compared to the niacin treatment.

Another aspect relates to a method for buffering a composition of a pharmaceutically acceptable niacin compound with a pharmaceutically acceptable L-carnitine compound. In a preferred embodiment, this method results in significantly reduced drug-induced skin flushing. In another embodiment, this method is accomplished without substantially causing drug-induced hepatotoxicity and/or other undesirable side effects. By reducing the levels of a pharmaceutically acceptable niacin compound required to be administered to a human, there may be less incidence of any side-effects of a traditional course of niacin, including but not limited to, skin flushing or irritation, hepatotoxicity, uric acid elevation, glucose level elevation, and peptic ulcers.

Another aspect relates to a method for the therapeutically effective prevention or treatment of cardiovascular disease including hyperlipidemia and atherosclerosis. Another embodiment is a method of preventing of reducing the incidence of myocardial infarctions, stroke, and peripheral vascular disease. In one embodiment of the method is when a composition comprising at least one active ingredient of the at least one pharmaceutically acceptable L-carnitine compound totals x moles and at least one active ingredient of the pharmaceutically acceptable niacin compound totals y moles and the mole ratio of x/y may be between 0.5 and 100 is administered to a human. Otherwise stated, the total moles of L-carnitine, whether present as L-carnitine or a salt, ester or derivative thereof, totals x moles and the total moles of niacin, whether present as nicotinic acid, niacinamide or other derivative or form, totals y moles and the mole ratio of x/y is preferably between 0.5 and 100. For the sake of clarity, one mole niacinamide equals one mole niacin, one mole ascorbyl niacinamide equals one mole niacin, and one mole of inositol hexanicotinate equals six moles niacin. In another embodiment, the mole ratio of x/y is between 0.6 and 76. In another embodiment, the mole ration of x/y is between 3.0 and 16. These embodiments may also have the effect of reducing the plasma concentration of a low density lipoprotein such as Lp(a) or LDL-cholesterol.

In some embodiments, the pharmaceutically acceptable L-carnitine compound and the pharmaceutically acceptable niacin compound are in a single composition. In other embodiments, the L-carnitine can be provided by one source and niacin can be provided by a different source. Thus, some methods are directed to administering the pharmaceutically acceptable L-carnitine compound and the pharmaceutically acceptable niacin compound in different compositions. Such different compositions may also be administered at different times.

Another embodiment provides an immediate release formulation comprising a pharmaceutically acceptable L-carnitine compound and a pharmaceutically acceptable niacin compound to reduce the plasma concentration of a low density lipoprotein without significant occurrences of skin flushing, skin irritation, or hepatotoxicity. Another embodiment provides a sustained release formulation comprising a pharmaceutically acceptable L-carnitine compound and a pharmaceutically acceptable niacin compound.

Another aspect relates to a treatment that reduces the plasma concentrations by administering multiple, daily doses of a composition comprising, or in some embodiments, consisting essentially of, a pharmaceutically acceptable L-carnitine compound and a pharmaceutically acceptable niacin compound to a human in order to reduce the amount of niacin intake to avoid side-effects of a regular treatment with niacin to reduce the plasma concentration of LDLs, including Lp(a).

### BRIEF DESCRIPTION OF THE DRAWINGS

FIG. 1 is a chart illustrating human tolerance of L-carnitine and niacin compositions.

### DETAILED DESCRIPTION OF THE INVENTION

Disclosed herein are compositions suitable for treating or preventing cardiovascular disease in a human comprising at least one pharmaceutically acceptable L-carnitine compound and at least one pharmaceutically acceptable niacin compound, wherein the at least one pharmaceutically acceptable L-carnitine compound and the at least one pharmaceutically acceptable niacin compound are in such amounts to reduce the plasma concentration of a low density lipoprotein in the human. Also disclosed are methods for using such compositions.

As used herein “lowering plasma concentration” or “to reduce the plasma concentration” means to lower the plasma concentration of an LDL. It may also mean to prevent the concentration of an LDL from being elevated. This includes, but is not limited to, Lp(a) and/or LDL-cholesterol.

As used herein, “pharmaceutically acceptable L-carnitine compound” includes the free base L-carnitine and/or any pharmaceutically acceptable salts and/or esters thereof. Pharmaceutically acceptable salts of L-carnitine include, in addition to the inner salts, all pharmaceutically acceptable salts which are prepared by the addition of acid to L-carnitine, and which preferably do not give rise to undesirable toxic or collateral effects. The formation of pharmaceutically acceptable acid addition salts is well known in pharmaceutical technology. Non-limiting examples of suitable salts include the chloride, orotate, acid aspartate, acid citrate, acid phosphate, fumarate, acid fumarate, lactate, maleate, acid maleate, acid oxalate, acid sulfate, glucose, phosphate, tartrate and acid tartrate salts. Likewise, ester preparation is well known in the art. Pharmaceutically acceptable esters of L-carnitine include, but are not limited to, acetyl-L-carnitine and propionyl-L-carnitine.

Suitable pharmaceutically acceptable L-carnitine compounds include, but are not limited to, any of the following: the free base of L-carnitine, L-carnitine tartrate, L-carnitine fumarate, L-carnitine succinate, L-carnitine maleate, L-carnitine malate, L-carnitine chloride, lysine-L-carnitine fumarate hydrochloride, acetyl-L-carnitine arginate dihydrochloride, acetyl-L-carnitine hydrogen fumarate, acetyl-L-carnitine taurinate hydrochloride, glycine propionyl-L-carnitine hydrochloride, creatine L-carnitine dihydrogen phosphate, L-omithine acetyl-L-carnitine dihydrochloride, creatine L-carnitine hydrogen fumarate, L-leucine acetyl-L-carnitine hydrochloride, or any mixture thereof.

As used herein, “pharmaceutically acceptable niacin compound” includes any compound that serves as a source for nicotinic acid, niacinamide, their anionic active ingredients, or mixtures thereof. Pharmaceutically acceptable niacin compounds include any pharmaceutically acceptable salt of nicotinic acid, niacinamide, or mixtures thereof. Examples include, but are not limited to, the following: nicotinic acid, niacinamide, inositol hexaniacinamide, ascorbyl niacinamide, inositol hexaniacinate, inositol nicotinate, vitamin B3. Niacin, as nicotinic acid or niacinamide, is converted in the body to the active forms, nicotinamide adenine dinucleotide (NAD) and a phosphorylated form (NADP) and the reduced forms NADH and NADPH.

As used herein, “consisting essentially of” means that while pharmaceutically acceptable L-carnitine and pharmaceutically acceptable niacin may be used in conjunction with excipients or other inactive components, these excipients and inactive components display no substantial effect on lowering the plasma concentration of a low density lipoprotein. As used herein, “a” when used with a component of the composition, dose, dosage form, and the like means “at least one”, that is, use of “a” does not, itself, require that there only be one.

As used herein, “cardiovascular diseases,” refers to disease states associated with high plasma levels of a low density lipoprotein. It includes, but is not limited to, arteriosclerosis, atherosclerosis, coronary artery disease, peripheral artery disease, myocardial infarction, stroke, restenosis, and bypass graft stenosis.

As used herein “active ingredient” may refer to the chemically active part of a compound. It may also refer to a group of chemically active parts of the same compound or multiple compounds. The potential active ingredients of L-carnitine may include L-carnitine in its cationic form, L-carnitine as an electrically neutral compound, and any esters of L-carnitine, including, but not limited to, acetyl-L-carnitine, propionyl-L-carnitine, or mixtures thereof. The potential active ingredients of niacin may include, but are not limited to, nicotinic acid, nicotinate, niacinamide, the anion of niacinamide, or mixtures thereof.

As used herein “to produce a mutual synergistic effect” may refer to an unexpected result that would not be foreseen by a person having ordinary skill in the art. For example, the buffering of a traditional niacin treatment with L-carnitine reduced the general side-effects of a human being treated with niacin. It may also mean a greater than additive effect. Thus, one explanation is that the combination of L-carnitine with niacinamide has a greater than expected result due to a mutual synergistic effect of the two active ingredients in lowering the plasma concentration of a low density lipoprotein.

As used herein “in such amounts to make the composition therapeutically effective” refers to an amount of a pharmaceutically acceptable L-carnitine compound and a pharmaceutically acceptable niacin compound, when administered to a human is sufficient to prevent or treat the elevation of plasma concentration of LDLs, preferably with reduced side-effects from niacin. This therapeutic amount can be determined routinely by a person having ordinary skill in the art in view of the disclosure herein.

As used herein “LDL-cholesterol” refers to cholesterol contained in LDL lipoprotein, including cholesterol contained in Lp(a).

A preferred embodiment is a composition comprising a pharmaceutically acceptable L-carnitine compound and a pharmaceutically acceptable niacin compound, in such amounts as to make the composition effective in reducing the plasma concentration of an LDL.

Another embodiment of the composition consists essentially of a pharmaceutically acceptable L-carnitine compound and a pharmaceutically acceptable niacin compound, in such amounts as to make the composition effective in reducing the plasma concentration of an LDL, and no other included ingredients may be present so as to have a substantial effect on the plasma concentration of a low density lipoprotein. In a preferred embodiment, this composition may reduce the plasma concentration of Lp(a). Another embodiment of the composition may reduce the plasma concentration of LDL-cholesterol. This composition may contain excipients as defined herein. This composition may also not substantially cause the significant side-effects of a traditional niacin regimen.

In another embodiment, the composition contains a certain mole ratio of the active ingredients of L-carnitine compounds to the active ingredients of niacin compounds. This mole ratio represents a proportion of active ingredients in the composition. Otherwise stated, the mole ratio is the total moles of L-carnitine, whether present as L-carnitine or a salt, ester or derivative thereof, to the total moles of niacin, whether present as nicotinic acid, niacinamide or other derivative or form. For the sake of clarity, one mole niacinamide equals one mole niacin, one mole ascorbyl niacinamide equals one mole niacin, one mole of inositol hexanicotinate equals six moles niacin, and a mixture of one mole of inositol hexanicotinate and one mole of niacinamide equals seven moles niacin. This proportion may vary to tune the effects that the composition may have on the plasma concentration of LDLs, preferably Lp(a). The proportion may also be varied to reduce, minimize, or negate any potential side-effects as a result of niacin intake and/or L-carnitine intake. These side-effects include, but are not limited to, flushing, hepatotoxicity, uric acid elevation, glucose elevation, peptic ulcers, and any other side-effect caused by niacin ingestion. L-carnitine generally does not have side-effects. However, a few reports have indicated that L-carnitine and its derivatives may cause increase in blood pressure, faster heartbeats, fever, or diarrhea. These side-effects, while rare, may also be avoided by tuning the proportion. The proportion and the effectiveness may also vary according to the human ingesting the composition. An embodiment of the composition is where the mole ratio of the active ingredients of L-carnitine compounds to the active ingredients of niacin compounds may be between 0.50 and 100. Another embodiment is where the mole ratio may be between 0.6 and 76. A preferred embodiment may be where the mole ratio is between 3.0 and 16.

As compared to a traditional niacin treatment, certain embodiments reduce the side effects typically associated with niacin ingestion. Thus, these embodiments do not substantially causes the side effects as acknowledged above. In embodiments, L-carnitine can provide a buffering effect for a person ingesting niacin or a pharmaceutically acceptable niacin compound. As compared to a typical niacin treatment, these embodiments substantially reduce the side effects associated with niacin or other pharmaceutically acceptable forms of niacin.

Another embodiment of a composition is one in which the L-carnitine and niacin components are in such amounts to produce a mutual synergistic effect in lowering LDL plasma concentration. A preferred embodiment is a composition that produces both a mutual synergistic effect by lowering LDL plasma concentration and substantially reduces side-effects associated with niacin treatment. A preferred embodiment may reduce the plasma concentration of Lp(a). Some embodiments include compositions of L-carnitine and niacin by themselves or in combination of any other additional nutritional ingredients, such as but not limited to vitamins and minerals commonly used in the manufacture and distribution of nutritional supplements to humans in the United States.

The method of administering a composition of a pharmaceutically acceptable L-carnitine compound with a pharmaceutically acceptable niacin compound may be accomplished by any means, although oral dosage is preferred. It may be accomplished by ingestion of a tablet, hard or soft capsules, powder, pill, drink, or lozenges. The formation of suitable oral dosage forms, may include, but are not limited to, those that are known to those having skill in the art. Compositions intended for oral use may be prepared according to any method, and such compositions may contain one or more agents such as sweetening agents, flavoring agents, coloring agents, and/or preserving agents in order to provide pharmaceutically elegant and palatable preparations.

The active ingredients in the admixture may also be combined with one or more non-toxic pharmaceutically-acceptable excipients which are suitable in the manufacture of capsules, tablets, pills, or powder. These excipients may be for example, inert diluents, such as calcium carbonate, sodium carbonate, lactose, calcium phosphate, or sodium phosphate; granulating and disintegrating agents, for example, corn starch, cellulose or modified celluloses or alginic acid; binding agents, for example, starch, cellulose or modified cellulose, gelatin, silica, or acacia; lubricating agents, for example magnesium stearate, calcium stearate, sucrose stearate, stearic acid, palmitic acid, ascorbyl palmitate or talc; or filling agents, for example, cellulose, methylcellulose, isopropylcellulose, ethylcellulose, isopropylmethylcellulose, or derivatives thereof. The oral dosage form may be uncoated or may be coated by techniques to delay disintegration and absorption in the gastrointestinal tract and thereby provide a sustained action over a longer period. Furthermore, dosage form may be by an immediate release, sustained release, or intermediate release formulation as prepared by a person having ordinary skill in the art.

Some embodiments may also include any other nutritional ingredients, such as, but not limited to vitamins and minerals commonly used in the manufacture and distribution of nutritional supplements to humans in the United States.

The invention is further described in terms of the following examples which are intended for the purpose of illustration and not to be construed as in any way limiting the scope of the present invention, which is defined by the claims.

### EXAMPLES

### Example 1

A composition was made by mixing 37.5 mg of inositol nicotinate, containing 33.9 mg of niacin with 5 mg cellulose, 1 mg silica, and 3 mg of stearic acid. This mixture was hand blended with another mixture consisting of 500 mg of L-carnitine-L-tartrate, containing 340 mg of L-carnitine, 6 mg of silica, 11 mg of magnesium stearate, and the resulting mixture was placed in a gelatin capsule (122 mg).

### Example 2

A composition was made by mixing 425 mg of L-carnitine fumarate, containing 248 mg of L-carnitine, with a mixture of 38 mg of inositol nicotinate, containing 33.9 mg of niacin, 5 mg of cellulose, 7 mg of silica, 8 mg of stearic acid, and 13 mg of magnesium stearate, and the resulting mixture was placed in a gelatin capsule (122 mg).

Another aspect relates to a method of administering to a human a composition comprising at least one pharmaceutically acceptable L-carnitine compound and at least one pharmaceutically acceptable niacin compound, in such amounts as to reduce the plasma concentration of a low density lipoprotein. Another embodiment of the method comprises administering to a human a composition comprising or in one embodiment, consisting essentially of, at least one pharmaceutically acceptable L-carnitine compound and at least one pharmaceutically acceptable niacin compound, in such amounts as to reduce the plasma concentration of a low density lipoprotein. In another preferred embodiment, such administration substantially reduces and/or eliminates side-effects of niacin treatment.

A preferred embodiment is the oral administration of the composition at least once daily. Another embodiment of the method comprises administering the composition in multiple doses per day. Dose may be defined as the amount given to a patient at any given time. Dose may also be administered over the course of a given time period. A daily dose may be administered over the course of several instant doses. Likewise, a weekly, monthly, or the like, dose may be administered over the course of several instant doses.

One embodiment is of a treatment with a composition comprising between 100-10000 mg of L-carnitine and 20-240 mg of niacinamide or nicotinic acid. Another embodiment includes treatment with a composition comprising between 200-500 mg of L-carnitine and 20-240 mg of niacinamide or nicotinic acid. The dosage can be determined by a person of skill in the art. The actual preferred amounts of the composition may vary according to various factors that are well known in the art, such as the particular composition formulated, the mode of ingestion, and the particular subject being treated. The dosage may depend on, inter alia, the mass of the person, the degree of reduction of the plasma concentration of a lipoprotein, a human's medical history, a human's current condition, a human's current dosage of other drugs, and a human's sensitivity to niacin treatment.

One embodiment of the method is where the composition can be administered in an amount that is both effective for reducing or preventing the elevation of plasma concentration of a low density lipoprotein, but does not substantially cause significant side effects of niacin treatment. Another embodiment would not substantially cause significant skin flushing. Another embodiment would not essentially cause any hepatotoxicity or would cause substantially no hepatotoxicity. Another embodiment would not generally cause elevated levels of glucose and/or uric acid in the individual being treated.

### Testing

One test has been performed on one subject. Additionally, tests have been performed on six additional subjects according to the following protocol to determine the effects of L-carnitine/niacin compositions. All subjects were between the ages of eighteen and forty-five, were in good health, had normal blood pressures, were non-smokers, had no previous medical conditions, and were taking no other prescription medications.

Three compositions were used to test each subject's response to treatment. The first composition “A” containing only 37.5 mg of niacin was encapsulated in a gelatin capsule with other non-active binders, lubricants, and fillers. A second composition “B” containing 37.5 mg niacin and 425 mg of L-carnitine tartrate, providing 285 mg of L-carnitine was encapsulated with inactive ingredients in a gelatin capsule. A third composition “C” containing inositol hexaniacinamide or inositol hexanicotinate equivalent to 37.5 mg of niacin, and 425 mg of L-carnitine fumarate, providing 245 mg L-carnitine, was encapsulated in a gelatin capsule with other inactive binders, lubricants, and fillers.

In the study on the single subject, the compositions were ingested orally, two capsules, three times daily. The first composition resulted in flushing and discomfort symptoms the subject. The second composition resulted in flushing only for the subject. The third composition resulted in no flushing or discomfort symptoms for the subject.

For the multi-subject study, for one week, each subject ingested two capsules of the first composition “A”, three times a day, for a total of six capsules per day. The total intake of niacinamide or niacin was 1125% higher than the normal recommended daily intake of niacin. Each subject recorded his responses to the drug in a daily questionnaire. After the first week, each subject ended this regimen and followed it with one week of no medication. After the end of the second week, each subject ingested two capsules of the second niacin composition “B”, three times a day, for a total of six capsules per day, for one week. Again, a daily record was made by each subject on his responses to the medication. At the end of the third week, the subject ended this regimen and followed it with one week of no medication. At the end of the fourth week, each subject ingested two capsules of the third composition “C”, three times a day, for a total of six capsules per day. At the end of the fifth week, the subject resumed his normal nutritional habits without any medication and the testing was ended. The responses were tabulated at the completion of each week.

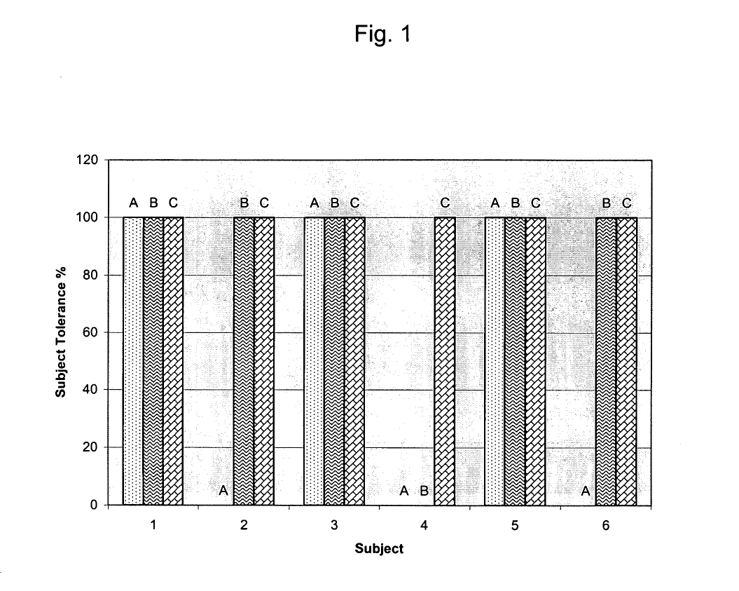

The results are presented in FIG. 1. Composition A resulted in flushing and discomfort symptoms for three of the six subjects. The second composition resulted in flushing and discomfort for one subject. The third composition resulted in no flushing or discomfort symptoms for any of the subjects.

The various compositions and methods described above provide a number of ways to carry out certain preferred embodiments of the invention. Of course, it is to be understood that not necessarily all objectives or advantages described may be achieved in accordance with any particular embodiment described herein. Thus, for example, those skilled in the art will recognize that the compositions may be made and the methods may be performed in a manner that achieves or optimizes one advantage or group of advantages as taught herein without necessarily achieving other objectives or advantages as may be taught or suggested herein.

Furthermore, the skilled artisan will recognize the interchangeability of various features from different embodiments. Similarly, the various components, features and steps discussed above, as well as other known equivalents for each such component, feature or step, can be mixed and matched by one of ordinary skill in this art to make compounds and perform methods in accordance with principles described herein.

Although the invention has been disclosed in the context of certain embodiments and examples, it will be understood by those skilled in the art that the invention extends beyond these specifically disclosed embodiments to other alternative embodiments and/or uses and obvious modifications and equivalents thereof. Accordingly, the invention is not intended to be limited by the specific disclosures of preferred embodiments herein.

What is claimed is:

1. A composition suitable for lowering plasma concentration of a low density lipoprotein in a human consisting essentially of:
at least one pharmaceutically acceptable L-carnitine compound; and
at least one pharmaceutically acceptable niacin compound,
wherein the at least one pharmaceutically acceptable L-carnitine compound and the at least one pharmaceutically acceptable niacin compound are present in amounts sufficient to reduce the plasma concentration of a low density lipoprotein in a human when administered orally.

2. The composition of claim 1, wherein the at least one pharmaceutically acceptable L-carnitine compound and the at least one pharmaceutically acceptable niacin compound are present in amounts to produce a mutual synergistic effect of lowering plasma levels of a low density lipoprotein in the human.

3. The composition of claim 1, wherein the at least one pharmaceutically acceptable L-carnitine compound comprises L-carnitine or a pharmaceutically acceptable salt and/or ester thereof.

4. The composition of claim 1, wherein
the at least one pharmaceutically acceptable L-carnitine compound is selected from the group consisting of the free base of L-carnitine, L-carnitine tartrate, L-carnitine fumarate, L-carnitine succinate, L-carnitine maleate, L-carnitine malate, L-carnitine chloride, lysine-L-carnitine fumarate hydrochloride, acetyl-L-carnitine arginate dihydrochloride, acetyl-L-carnitine hydrogen fumarate, acetyl-L-carnitine taurinate hydrochloride, glycine propionyl-L-carnitine hydrochloride, creatine L-carnitine dihydrogen phosphate, L-ornithine acetyl-L-carnitine dihydrochloride, creatine L-carnitine hydrogen fumarate, L-leucine acetyl-L-carnitine hydrochloride, and mixtures thereof; and
the at least one pharmaceutically acceptable niacin compound is selected from the group consisting of nicotinic acid, niacinamide, inositol hexaniacinamide, inositol hexaniacinate, ascorbyl niacinamide, and mixtures thereof.

5. The composition of claim 1, wherein the ratio of the total number of moles of the pharmaceutically acceptable L-carnitine compounds and the total moles of the pharmaceutically acceptable niacin compounds is about 0.50 to approximately 100.

6. The composition of claim 1, wherein the low density lipoprotein comprises Lp(a).

7. The composition of claim 6, wherein the amounts of the at least one pharmaceutically acceptable L-carnitine compound and the at least one pharmaceutically acceptable niacin compound are sufficient to cause a reduction in the plasma concentration of Lp(a) by at least 10%.

8. The composition of claim 1, wherein the low density lipoprotein comprises LDL-cholesterol.

9. The composition of claim 1, wherein the composition is in form of a tablet, caplet, capsule, powder, or other oral dosage form.

10. The composition of claim 1, wherein the composition includes one or more vitamins and/or minerals.

11. A composition suitable for lowering plasma concentration of a low density lipoprotein in a human comprising:
one or more pharmaceutically acceptable L-carnitine compounds present at a combined total of x moles (as moles of L-carnitine); and
one or more pharmaceutically acceptable niacin compounds present at a combined total of y moles (as moles of niacin),
wherein the mole ratio of x/y is approximately 0.50 to approximately 100.

12. The composition of claim 11, wherein the mole ratio of x/y is approximately 0.60 to approximately 76.

13. The composition of claim 11, wherein the mole ratio of x/y is approximately 3.0 to approximately 16.

14. The composition of claim 11, wherein the at least one pharmaceutically acceptable L-carnitine compound comprises L-carnitine or a pharmaceutically acceptable salt and/or ester thereof.

15. The composition of claim 11,
wherein the at least one pharmaceutically acceptable L-carnitine compound is selected from a group consisting of the free base of L-carnitine, L-carnitine tartrate, L-carnitine fumarate, L-carnitine succinate, L-carnitine maleate, L-carnitine malate, L-carnitine chloride, lysine-L-carnitine fumarate hydrochloride, acetyl-L-carnitine arginate dihydrochloride, acetyl-L-carnitine hydrogen fumarate, acetyl-L-carnitine taurinate hydrochloride, glycine propionyl-L-carnitine hydrochloride, creatine L-carnitine dihydrogen phosphate, L-ornithine acetyl-L-carnitine dihydrochloride, creatine L-carnitine hydrogen fumarate, L-leucine acetyl-L-carnitine hydrochloride, and mixtures thereof; and
wherein the at least one pharmaceutically acceptable niacin compound is selected from the group consisting of nicotinic acid, niacinamide, inositol hexaniacinamide, inositol hexaniacinate, ascorbyl niacinamide, and mixtures thereof.

16. The composition of claim 11, wherein the composition includes one or more vitamins and/or minerals.

17. The composition of claim 11, wherein the at least one pharmaceutically acceptable L-carnitine compound comprises the free base of L-carnitine, acetyl-L-carnitine, propionyl-L-carnitine, or any mixture thereof.

18. The composition of claim 11, wherein the at least one pharmaceutically acceptable niacin compound comprises nicotinic acid, niacinamide, or a mixture thereof.

19. The composition of claim 11, wherein the composition is in form of a tablet, caplet, capsule, powder, or other oral dosage form.

20. A method of reducing plasma concentration of a low density lipoprotein in a human comprising:
administering at least once daily a pharmaceutical composition consisting essentially of at least one pharmaceutically acceptable L-carnitine compound and at least one pharmaceutically acceptable niacin compound;
wherein total amounts of the at least one pharmaceutically acceptable L-carnitine compound and the at least one pharmaceutically acceptable niacin compound administered daily are therapeutically effective by lowering the plasma concentration of a low density lipoprotein in a human.

21. The method of claim 20, wherein the total amount of the at least one pharmaceutically acceptable L-carnitine compound is about 200-5000 mg/day.

22. The method of claim 20, wherein the total amount of the at least one pharmaceutically acceptable niacin compound is about 50-240 mg/day.

23. The method of claim 20, wherein the composition is administered in multiple doses and the at least one pharmaceutically acceptable niacin compound is administered at about 25-120 mg/dose.

24. The method of claim 20, wherein the at least one pharmaceutically acceptable L-carnitine compound comprises L-carnitine or a pharmaceutically acceptable salt and/or ester thereof.

25. The method of claim 20, wherein
the at least one pharmaceutically acceptable L-carnitine compound is selected from the group consisting of the free base of L-carnitine, L-carnitine tartrate, L-carnitine fumarate, L-carnitine succinate, L-carnitine maleate, L-carnitine malate, L-carnitine chloride, lysine-L-carnitine fumarate hydrochloride, acetyl-L-carnitine arginate dihydrochloride, acetyl-L-carnitine hydrogen fumarate, acetyl-L-carnitine taurinate hydrochloride, glycine propionyl-L-carnitine hydrochloride, creatine L-carnitine dihydrogen phosphate, L-ornithine acetyl-L-carnitine dihydrochloride, creatine L-carnitine hydrogen fumarate, L-leucine acetyl-L-carnitine hydrochloride, and mixtures thereof; and
the at least one pharmaceutically acceptable niacin compound is selected from the group consisting of nicotinic acid, niacinamide, inositol hexaniacinamide, inositol hexaniacinate, ascorbyl niacinamide, and mixtures thereof

26. The method of claim 20, wherein the ratio of the total number of moles of the pharmaceutically acceptable L-carnitine compounds and the total moles of the pharmaceutically acceptable niacin compounds is about 0.50 to approximately 100.

27. The method of claim 20, wherein the low density lipoprotein comprises Lp(a).

28. The method of claim 27, wherein the amounts of the at least one pharmaceutically acceptable L-carnitine compound and the at least one pharmaceutically acceptable niacin compound are sufficient to cause a reduction in the plasma concentration of Lp(a) by at least 10%.

29. The method of claim 20, wherein the low density lipoprotein comprises LDL-cholesterol.

30. A method of reducing flushing in a human being treated for hyperlipidemia with niacin or a derivative thereof comprising:
administering at least once daily a composition consisting essentially of:
one or more pharmaceutically acceptable L-carnitine compounds; and
one or more pharmaceutically acceptable niacin compounds,
wherein the one or more pharmaceutically acceptable L-carnitine compounds and the one or more pharmaceutically acceptable niacin compounds are present in such amounts to reduce plasma concentration of a low density lipoprotein and to cause substantially no niacin induced flushing.

31. The method of claim 30, wherein the total amount of the one or more pharmaceutically acceptable L-carnitine compounds administered is about 200-5000 mg/day.

32. The method of claim 30, wherein the total amount of the one or more pharmaceutically acceptable niacin compounds administered is about 50-240 mg/day.

33. The method of claim 30, wherein the composition is administered in multiple doses and the total amount of the one or more pharmaceutically acceptable niacin compounds administered in a single dose is about 25-120 mg/dose.

34. The method of claim 30, wherein treatment causes substantially no niacin induced hepatotoxicity.

35. The method of claim 30, wherein treatment does not cause any significant niacin induced elevation of glucose and/or uric acid concentration.

36. A method of treating hyperlipidemia comprising:
administering one or more pharmaceutically acceptable L-carnitine compounds present at a combined total of x moles (as moles of L-carnitine); and one or more pharmaceutically acceptable niacin compounds present at a combined total of y moles (as moles of niacin),
wherein the mole ratio of x/y is approximately 0.50 to approximately 100.

37. The method of claim 36, wherein the mole ratio of x/y is approximately 0.60 to approximately 76.

38. The method of claim 36, wherein the mole ratio of x/y is approximately 3.0 to approximately 16.
